(19) United States
(12) Patent Application Publication
(10) Pub. No.: US 2021/0267796 A1
(43) Pub. Date: Sep. 2, 2021

(54) OPHTHALMIC INSTRUMENT

(71) Applicant: Duckworth and Kent Limited, Baldock (GB)

(72) Inventors: Stephen Paul Woods, Hitchin (GB); David Christopher Hall, Letchworth Garden City (GB)

(21) Appl. No.: 17/185,207

(22) Filed: Feb. 25, 2021

(30) Foreign Application Priority Data
Feb. 28, 2020 (GB) 2002913.8

(51) Int. Cl. A61F 9/00; A61F 9/007; A61B 90/70; B08B 9/032

(52) U.S. Cl. CPC A61F 9/0017; A61F 9/007; B08B 2209/032; B08B 9/0323; B08B 9/0325; A61B 90/70

(57) ABSTRACT

The present invention provides for a squeeze handle for an ophthalmic instrument having elongate body with resiliently-biased squeeze elements, wherein a conduit is provided within the elongate body to allow for fluid communication between an opening provided in an outer wall of elongate body and a tool to be in operative engagement with the handle and the squeeze elements, and to a related method of cleaning an ophthalmic instrument having resiliently biased squeeze elements on an elongate handle body, and including the steps of introducing, by way of an opening provided in an outer wall of the elongate body, fluid under pressure into a conduit provided within the elongate body; and allowing for flow of the fluid along the conduit to a tool in operative engagement with the elongate handle and squeeze elements; for cleaning the tool.

### CLAIM OF PRIORITY UNDER 35 U.S.C. § 119

The present Application for Patent claims priority to Great Britain Patent Application No. 2002913.8 entitled “Ophthalmic Instrument” filed Feb. 28, 2020 and assigned to the assignee hereof and hereby expressly incorporated by reference herein.

### BACKGROUND

### Field

The present invention relates to ophthalmic instruments and, in particular, to a squeeze handle for an ophthalmic instrument, and an ophthalmic instrument including a squeeze handle, and to an arrangement and method of introducing fluid to the instrument for cleaning purposes; and other requirements such as the local delivery of fluid substances into a patient's eye as required. Delivery to, and/or from, a tool of the instrument is a particular aspect of the invention.

### Background

Ophthalmic instruments by their very nature comprise relatively small, high-precision, devices made to demanding tolerances. These features, allied with their inherent high cost, and the increasing relevance of environmental considerations, does not lend such devices to single-use, or even short lifetime, and disposability.

There is therefore a strong desire to clean, and sterilise, such instruments for purposes of longevity and reuse.

However, in view of the high precision of such instruments, and their necessity for high quality small parts, the instruments can be easily damaged during use, handling and manipulation. In addition, and again through necessity, such instruments often include/comprise sharp tool implements which introduce an inherent danger of ‘needle-stick’ injury for operatives such as theatre technicians when the instruments are required to be manipulated for cleaning purposes. This is particularly so when the manipulation involves de-mounting/separation of parts, such as for example separation of a tool element from a handle portion of the instrument.

Automated cleaning processes can be seen as advantageous for purposes of speed and to allow for batch cleaning. However, even then the nature of required manipulation can still expose the high precision tool element to unintentional and undesired impact and damage. Still further, operatives such as theatre technicians remain exposed to the potential of ‘needlestick’ type injuries.

Eye Technology Ltd have proposed a ‘VR Flushing Tool’ for attempted flush-cleaning of Vitreoretinal Instruments in which the complete instrument, including handle and tool, is encased within a barrel of the flushing tool, and flushing fluid then introduced into the barrel at a pressure high enough to allow for ingress of the fluid into, and out of, the instrument through apertures that exist in its structure at the point of connection/mounting of the moveable elements of the tool. However, this requires high pressures and there is a disadvantageous uncertainty as to the effectiveness of the flushing/cleaning process. There is therefore a danger that the instrument might pass on to its sterilisation phase with contaminant still in situ, and the attempted sterilisation will only serve to retain the contaminant rendering the instrument useless.

The likelihood of damage and personal injury is still therefore a disadvantageously likely factor when looking to clean reliably ophthalmic instruments for re-use.

There is also a desire to allow for a time saving, yet accurate, injection of fluid substances into the eye, and ideally at an optimal location for maximal effect and at a minimal amount/concentration.

### SUMMARY

The present invention seeks to provide for an ophthalmic instrument, and in particular a squeeze handle ophthalmic instrument having advantages over known such instruments.

In particular, the invention seeks to provide for a squeeze handle for an ophthalmic instrument, whether multipart or unitary, having advantages over known such handles and instruments.

The invention also seeks to provide for a method of cleaning such instruments and the tools associated therewith; and to a method for the delivery of fluid substances into the eye, and at optimal location for maximal effect and at minimal amount/concentration.

According to one aspect of the present proposal there is provided a squeeze handle for an ophthalmic instrument having an elongate body with resiliently-biased squeeze elements, wherein a conduit is provided within the elongate body to allow for fluid communication between an opening provided in an outer wall of elongate body and a tool when in operative engagement with the handle and the squeeze elements.

The squeeze elements can be provided in the region of an end of the elongate body to be proximate to the tool.

Advantageously, the opening can be provided in the region of an end of the elongate body to be distal to the tool.

In particular, the opening can be provided at the distal end of the elongate body.

The conduit can extend along at least the substantial length of the elongate body.

Advantageously, the conduit can extend along the full length of the elongate body.

As a further feature, the conduit can extend at least substantially co-axially along at least a substantial part of the elongate body. In particular, the opening can have an engagement formation configured to engage with an output of a fluid delivery arrangement. Examples of such formations can comprise fittings such as integrated luer lock fittings.

Yet further, the conduit can advantageously be arranged/configured to deliver fluid to within at least part of the tool.

Still further, the conduit can further be configured to be contiguous with, and deliver fluid to, a conduit found within the tool.

Advantageously the conduit in the body can be configured to extend coaxially within the conduit within the tool. In particular, the elongate body can be arranged for the replaceable mounting of a tool.

Of course, the tool can be provided in a manner integral with elongate body.

In one arrangement the handle is arranged to receive a cleaning fluid which in one example will serve to flush the instrument, and in particular the tool of the instrument.

In another arrangement, the handle is arranged to receive fluid substances for onward delivery via the instrument into the eye, and ideally at an optimal location for maximal effect and at minimal amount/concentration.

The squeeze handle of the invention can also include, either as an integral or mounted element, such as by way of a press-fit configuration, a tool mount portion in fluid communication with the elongate body and for receiving the tool.

At least part of the tool mount portion can be movable relative to the elongate body for actuation of the tool. In this manner the tool mount portion can comprise a multipart feature of the invention.

As an alternative, the tool mount portion can comprise a unitary member arranged to be movable relative to the elongate body for actuation of the tool.

The width of a path for the fluid communication decreases at least once in the direction of fluid flow.

In particular, the squeeze handle can be configured such that the at least one decrease in the path width is in the region of the tool. Such a decrease advantageously serves to increase the pressure exhibited by the flowing fluid at the location of the decrease which can then be at or near the tool to assist with the flushing thereof.

As will be readily appreciated the invention can provide for a one-piece ophthalmic instrument including a squeeze handle as outlined above.

According to another aspect of the present invention there is provided a method of cleaning an ophthalmic instrument having resiliently biased squeeze elements on an elongate handle body, and including the steps of:
a. introducing, by way of an opening provided in an outer wall of the elongate body, fluid under pressure into a conduit provided within the elongate body; and,
b. allowing for flow of the fluid along the conduit to a tool in operative engagement with the elongate handle and squeeze elements; for
c. cleaning the tool.

According to one particular advantage, the method can include the step of directing the fluid introduced into the conduit into an inner part of the tool.

Yet further, the method can also include the step of flushing the tool by way of the fluid as introduced.

Of course, it will be appreciated that the method can include the step of mounting the ophthalmic instrument by way of the opening in the elongate body onto a fluid delivery arrangement.

Yet further, the method can include the step of mounting the ophthalmic instrument onto a nozzle of a fluid delivery arrangement.

Of course, it will also be appreciated that the method can form at least part of an automated cleaning process.

Still further, the invention can provide for an ophthalmic instrument sterilisation apparatus, including a fluid delivery mechanism having at least one nozzle arrange to engage with the opening in the body of a squeeze handle as defined above.

In particular, the nozzle is arranged for the mounting of the ophthalmic instrument within the apparatus. The apparatus may include a plurality of nozzles for the receipt of a plurality of ophthalmic instruments. The apparatus can also present a housing within which the nozzles are located. As a further feature, the apparatus can be arranged to exhibit control functionality allowing for an at least part-automated cleaning process.

According to yet another aspect of the present invention there is provided a method for the introduction of a fluid substance into a patient's eye, by way of an ophthalmic instrument having resiliently biased squeeze elements on an elongate handle body and including the steps of:
a. introducing, by way of an opening provided in an outer wall of the elongate body, the fluid substance into a conduit provided within the elongate body; and,
b. allowing for flow of the fluid along the conduit to a tool in operative engagement with the elongate handle and squeeze elements; for
c. delivery of the fluid substance to the patient's eye via the tool.

This aspect of the invention is particularly advantageous, in so far as the conduit can be readily employed to allow for the injection of fluid substance(s) into a patient's eye at optimal location for maximal effect and minimal amount/concentration, and in a particularly efficient and reliable manner.

### BRIEF DESCRIPTION OF THE DRAWINGS

The invention is described further hereinafter, by way of example only, with reference to the accompanying drawings in which:

FIG. 1 is a side view of a squeeze handle ophthalmic instrument according to an embodiment of the present invention;

FIG. 2 is a perspective view of the instrument illustrated in FIG. 1;

FIG. 3 is a sectional view along the longitudinal axis of the instrument and in the same direction of the view as in FIG. 1;

FIG. 4 is a sectional view similar to that of FIG. 3, but with the handle is a squeezed configuration so as to activate the tool;

FIG. 5 is a side view of a squeeze handle ophthalmic instrument according to another embodiment of the present invention;

FIG. 6 is a sectional view along the longitudinal axis of the instrument of, and in the same direction of the view as in, FIG. 5;

FIG. 7 is a sectional view similar to that of FIG. 6, but with the handle is a squeezed configuration so as to activate the tool; and

FIG. 8 is a partial perspective view of flushing apparatus arranged for the simultaneous cleaning of a plurality of instruments.

### DETAILED DESCRIPTION

It should of course be appreciated that the concept of the present invention is applicable to any form of squeeze handle ophthalmic instrument whether or not that instrument might comprise a multi-part instrument, with replaceable parts, or a ‘single piece’ instrument formed of integral elements.

Also, the nature of the tool provided by way of the instrument is not restricted to that of the illustrated embodiment and can comprise any appropriate functional/operative/surgical element requiring cleaning and subsequent sterilisation to allow for reuse; and/or to be employed for the delivery of a fluid substance into a patient's eye.

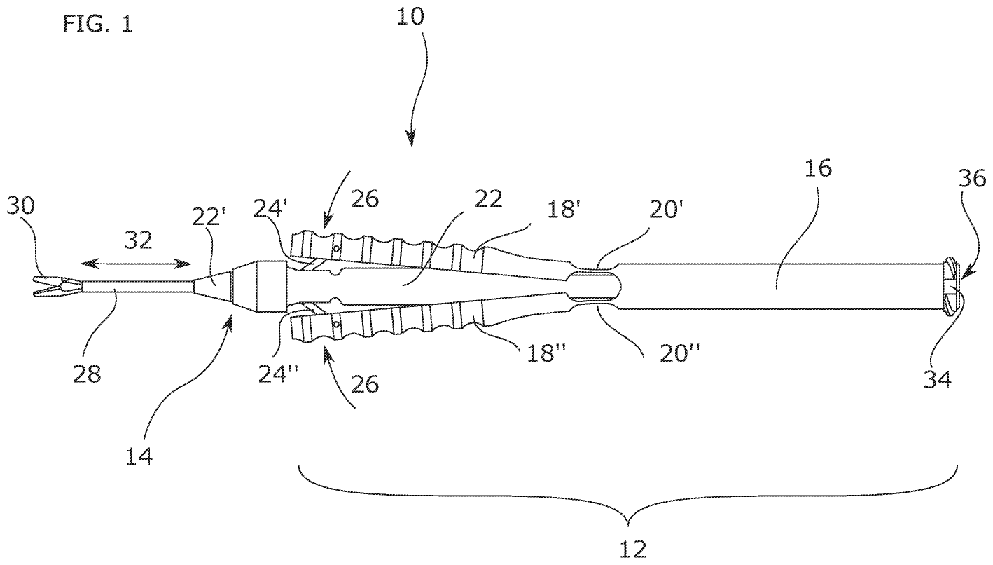

Turning now to FIG. 1, there is illustrated a side view of a squeeze handle ophthalmic instrument 10 having a handle portion 12 and a tool portion 14 operatively mounted in engagement with the handle portion 12.

The handle portion 12 includes an elongate cylindrical handle body 16 having resiliently biased squeeze elements 18′, 18″ extending outwardly, in view of their bias, at an acute angle from the longitudinal axis of the body 16. In the illustrated example, the resilience is provided by thinning of the dimensions of each of the elements 18′, 18″ at respective spring neck regions 20′, 20″, and at which the squeeze elements 18′, 18″ effectively pivot in a resilient manner relative to the body 16.

The tool of the instrument is provided integral with the body 16 by means of a cylindrical tool mount 22 inserted to a secure press-fit within the body 16. Respective linkage arms 24′, 24″ are pivotally mounted to the underside of each of the squeeze elements 18′, 18″ and during a squeeze motion of the elements 18′, 18″ in the direction of arrows 26, the linkage arms 24′, 24″ which are also in operative engagement with the tool via the tool mount, serve to activate the tool for the required operation (as described further below).

The tool portion 14 of the illustrated embodiment includes a tool tube 28 from which extends a scissor tip device 30 to be operated as required as part of an ophthalmic surgical procedure through use of the instrument 10. The tool tube 29 can be mounted to the tool mount 22 as a separate element, or integral therewith as an extension thereof.

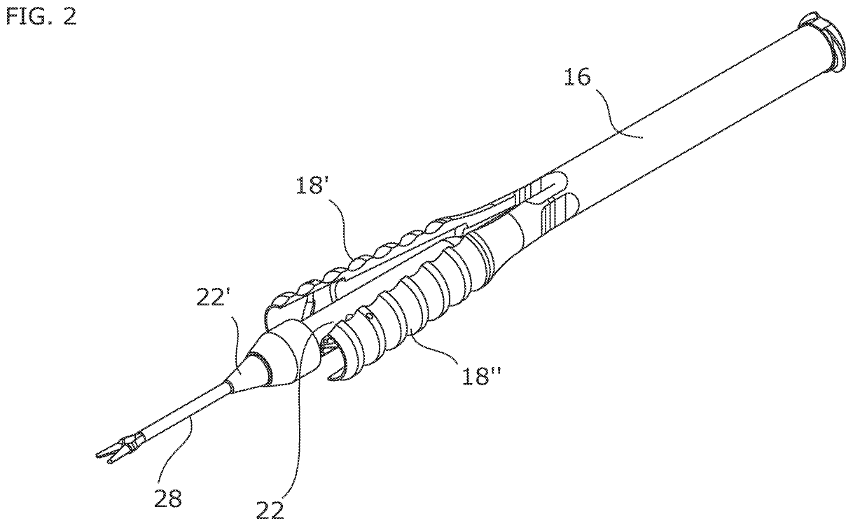

FIG. 2 is a perspective view of the instrument of FIG. 1 to provide further detail of its configuration.

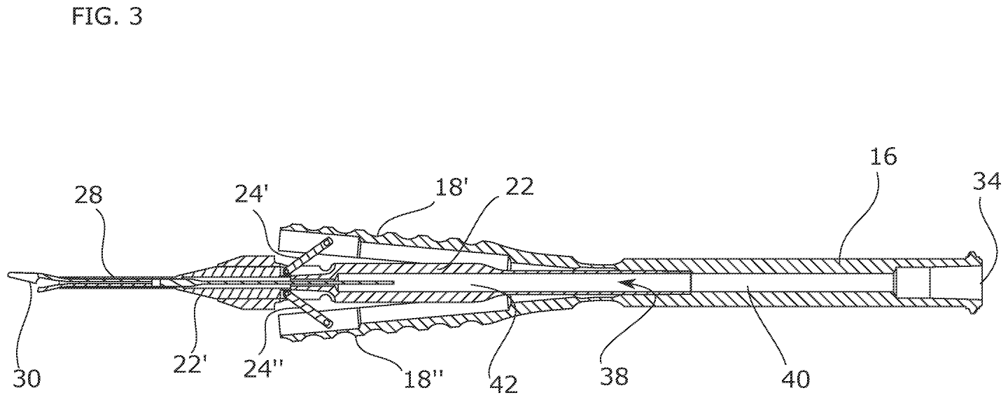

In the illustrated example, the squeeze action 26 of the squeeze elements 18′, 18″ serves to move the tool tube 28 in a reciprocal motion in the direction of arrow 32 as the squeeze elements 18′, 18″ are squeezed and then released under their bias. As illustrated further by FIG. 3 as discussed below, the cylindrical tool mount 22 can be provided as a two-part element 22, 22′ in which the more forward part 22′ engages with the linkage arms 24′, 24″ to move under influence of the squeeze action of the squeeze elements 18′, 18″, and to move the tool tube 28 in the direction of the scissor tip device 30 for the activation thereof. In the illustrated example, the tool tube 28 effectively forms a sleeve which moves relative to the scissor tip device 32, to encompass the scissor tip device 32, initiating a scissor action of the tip device 32, and thus allowing for the required surgical action.

As will be appreciated, such manner of use of the illustrated ophthalmic instrument 10 will require complete cleaning and sterilisation to allow for reuse of the instrument. The tolerances of the tool portion, and its operative parts 28, 30, make such cleaning and sterilisation procedures challenging. The concept of the present invention and as embodied, as one of many possible different examples, within the instrument 10 illustrated in the accompanying drawings, allows for such efficient and complete cleaning.

At the heart of the present invention, is a configuration for the elongate handle 16 of the instrument 10, that readily permits the required degree of certainty and efficiency for the cleaning process by allowing a thorough washing/flushing of the tool mount 22 and in particular the operative parts 28, 30 of the tool portion 14.

For an alternative operation, the configuration of the handle and instrument is to allow for delivery of a fluid substance into a patient's eye as part of an investigative or treatment procedure.

As described with further reference to the cross-sectional view provided by FIG. 3, the elongate cylindrical body 16 of the instrument 10 includes an opening 34 provided at its end 36 distal to the tool portion 14, and through which fluid can be introduced to the instrument 10 for eventual washing/flushing of the operative parts 28, 30 of the tool portion 14; and/or delivery of a fluid substance into a patient's eye as required.

As illustrated in FIG. 3, a coaxial passage 38 extends along the full length of the instrument 10, and in the illustrated embodiment actually also extends to the tool tube 28 and tool 30 itself. The passage 38, starts at the aforementioned opening 34 at the distal end 36 of the elongate cylindrical body 16, and comprises a conduit 40 within the body 16 which is contiguous with a conduit 42 within the tool mount 22, which is in turn contiguous with the inside of the tool tube 28.

As noted above in relation to FIG. 1, the cylindrical tool mount 22 has a separate forward part 22′ which moves in reciprocal manner of arrow 32 under the influence of the linkage arms 24′, 24″ and the squeeze elements 18′. 18″. The forward part 22′ moves in a guided manner within a sheath formation of the mount 22, and is connected to the tool tube 28 to urge the latter in the direction of the scissor tip device 30 to initiate the operation thereof. As an alternative, the tool tube 28 can be formed integral of the forward part 22′ of the mount 22 as an extension thereof. The tool 30 extends back through the tool tube 28 and is anchored, importantly within the passage 38, to the tool mount 22 to allow for relative motion between the tool tube 28 and tool 30.

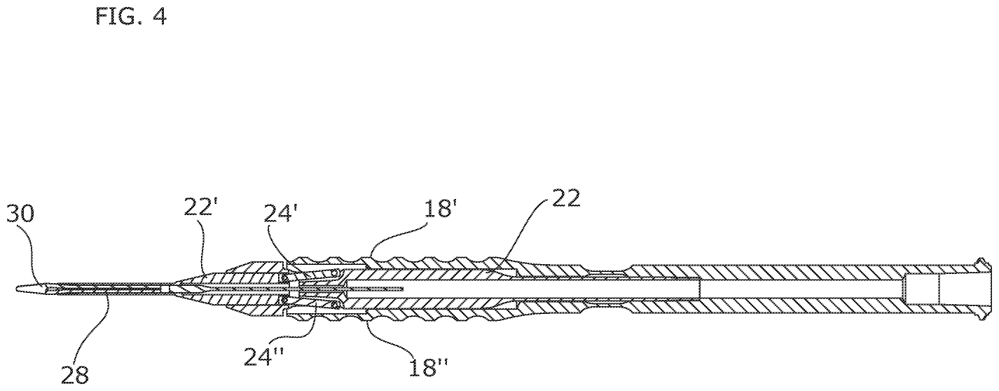

FIG. 4, which illustrates the device of FIGS. 1-3 with the elements 18′, 18″ depressed, or squeezed, clearly illustrates how the linkage arms have urged the forward part 22′ forward of the mount 22 and handle body 16 and such that the tool tube 28 is likewise moved so as to effectively envelop the scissor tip device 30 so as to close the tip, i.e. activate the tool.

As will therefore clearly be appreciated from FIG. 3, fluid introduced under pressure at the opening 34 of the body 16 will pass through the instrument and into the tool tube 28 from where it will exit at the scissor tip 30. FIG. 3 also illustrates the stepwise decrease in diameter of each of the aforementioned conduits 40, 42 and tool tube 28. This serves to increase the pressure of the fluid as it passes through the instrument 10 to thereby assist with the cleaning/flushing process at the tool portion 14. In addition, the fluid also serves to clean all inner regions/surfaces of the instrument and such as those associated with the spring action of the spring elements 18′, 18″, and the linkage arms 24′, 24″ and their connections of these elements within the instrument.

In particular, the resilient bias offered by the squeeze elements 18′, 18″ and connectivity within the instrument 10 provided by the associated linkage arms 24′, 24″, advantageously serves as a pressure release valve. That is, if the flushing pressure of the fluid within the instrument 10 exceeds a certain level, movement of the tool tube 28 (as usually initiated by squeezing the squeeze elements 18′, 18″) will occur. This not only allows for a pressure-relief function, but also serves to move the movable elements within the instrument and associated with the squeeze elements. This in turn can serve to open a gap within the fluid pathway through which some fluid can escape so as to ensure full cleaning/washing of, for example, the linkage elements 24′, 24″ within the illustrated example.

All operative/movable parts within the instrument can therefore be cleaned to an advantageously high degree of effectiveness and reliability.

The present invention also provides for a method of cleaning an ophthalmic instrument, and it will be appreciated that the illustrated embodiment provides one example of an instrument that can really benefit from such a method.

That is, the opening 34 at the distal end 36 of the elongate body 16 of the instrument 10 can readily be employed for the step of efficiently introducing fluid into the instrument. In particular, if the method is envisaged as part of an automated process, the opening 34 can readily comprise, or be provided with, an appropriate engagement formation, by means of which the handle portion 12 of the instrument can be mounted for receiving the fluid under pressure as part of such an automated process. Examples of such formations can comprise fittings such as integrated luer lock fittings.

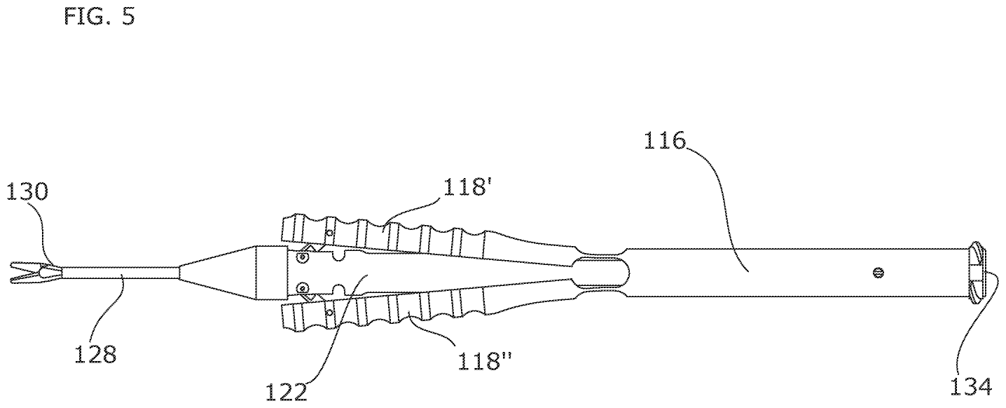

An alternative embodiment of the invention is illustrated with reference to FIGS. 5 to 7.

As discussed further, while a different configuration for tool mounting and operation is noted, the configuration of the whole instrument, and in particular the handle portion and its conduit, is such that the advantageous flushing and/or fluid delivery of the invention remains.

Turning first to FIG. 5, this alternative embodiment again comprises a handle portion 116 with an opening 134 for the delivery of fluid. A pair of oppositely disposed squeeze elements 118′, 118″ extend from the handle portion 116 for use in moving a tool tube 128 in the direction of a scissor tip device 130 and for the activation thereof.

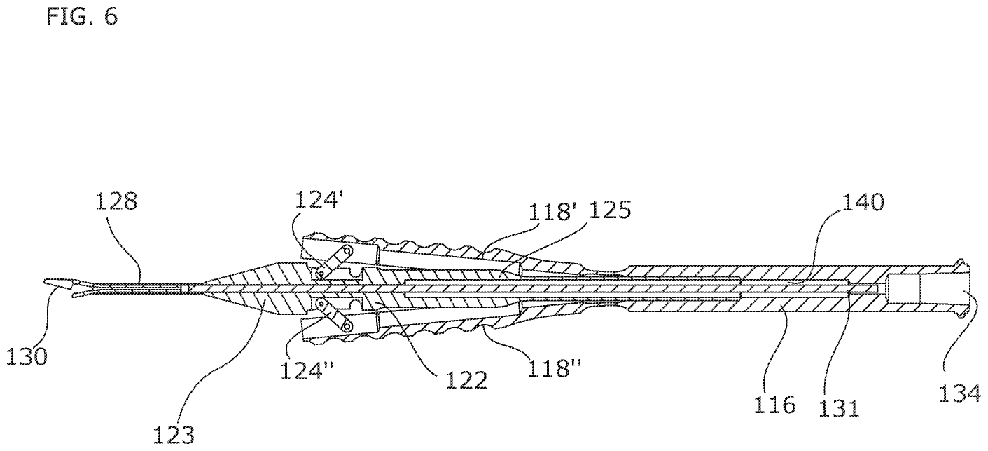

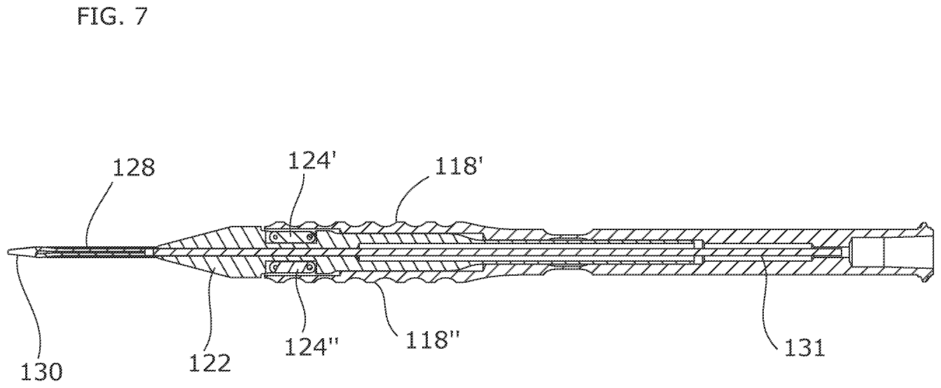

However, in this embodiment, and as illustrated further with reference to FIGS. 6 and 7, the cylindrical tool mount 22 is provided as a one-piece element. Referring first to FIG. 6, there is further shown linkage arms 124′, 124″ operatively connecting the squeeze elements 118′, 118″ to a forward portion 123 of the unitary tool mount 122. In view of such unitary construction, it is the whole tool mount 22 that moves under the influence of the squeeze elements 118′, 118″.

As clear from FIG. 6, the tool mount 122 also includes a rear portion 125 exhibiting a tail 127 that extends back into the conduit of the handle body 116. The tail 127 is arranged for sliding motion against the inner wall of the handle body 116 forming its conduit. This enhances the stable/guided motion of the tool mount 122 as initiated by operation of the squeeze elements 118′, 118″. The moving tool mount 122 likewise urges the tool tube 128 in the direction of the scissor tip device 130 for operation thereof. The scissor tip device 130 is integral with, or connects to, a tool anchor 131 which extends back through the handle body and is secured thereto, for example by way of a clamping screw (not sown in FIG. 6). This ensures the relative motion between the tool mount 122/tool tube 128 and the scissor tip device 130 required for operation of the scissor tip device 130. It should be appreciated that such anchorage mechanism does not interfere with the flow of fluid through the conduit of the handle body 116 as required by the invention.

The cross-sectional view of FIG. 7 shows the squeeze elements 118′, 118″ depressed/squeezed and having moved the tool mount 122 in its guided manner within the handle body 116 so as to activate the scissor tip device 130 by way of the tool tube 128.

Within this further embodiment also therefore, a coaxial passage extends along the full length of sliding tool mount 122. Starting at opening 134 at the distal end of elongate handle body 116, the passage comprises a conduit 140 within the handle body 116 which is contiguous with a conduit within the sliding tool mount 122. Fluid introduced under pressure at the opening 134 of the handle body 116 will pass through the instrument until exiting at the scissor tip device 130. The diameter of the aforementioned conduit within the sliding tool mount 122 decreases at the proximal end to increase the pressure within the fluid as it passes through the instrument to thereby assist with the cleaning/flushing process of the scissor tip device 130.

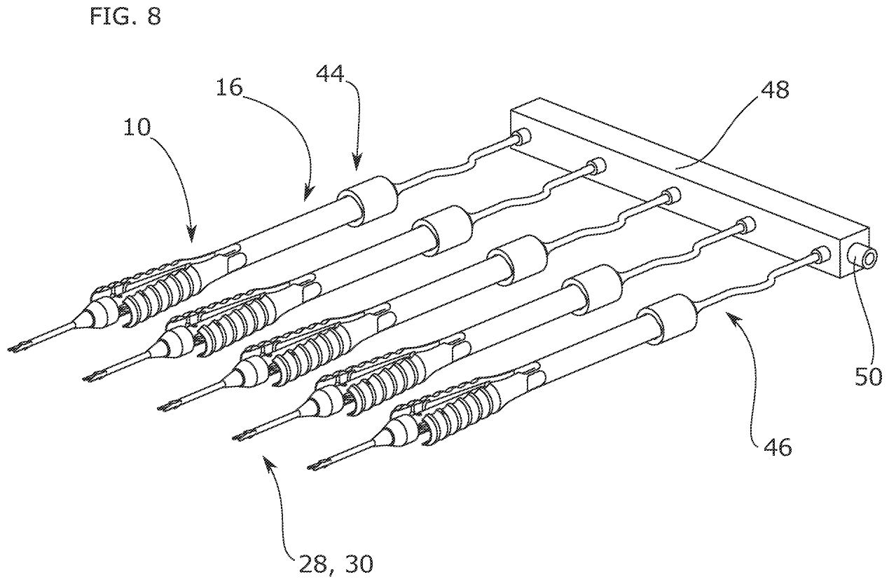

In FIG. 8 there is illustrated a partial perspective view of an arrangement within an apparatus for the simultaneous mounting and flushing/cleaning of a plurality of instruments such as those discussed with reference to FIGS. 1-4 of the preceding drawings. It should be appreciated that the features illustrated in FIG. 8 can be mounted and operatively connected within any appropriate housing/chamber (not shown in the drawing) of the apparatus to allow for an at least part-automated cleaning process.

Referring to FIG. 8, five instruments 10 such as those discussed above in relation to FIGS. 1-4 are illustrated as connected in parallel to respective nozzle couplings 44, which are arranged to provide for a fluid-tight coupling for the delivery of a cleaning fluid into the conduits within the handles 16. The distal ends of the handles 16 are mounted to the nozzle couplings 44 by any appropriate engagement formations allowing for a press-fit, or twist coupling such as by engaging screw threads. Examples of such formations can comprise fittings such as integrated luer lock fittings. Within the apparatus the cleaning fluid is delivered to the nozzle couplings 44 by respective delivery pipes 46 leading from a manifold 48 which is itself fed by way of a manifold inlet 50 by a supply of cleaning fluid. As will be appreciated, the flushing/cleaning process simply requires the simple location and mounting of each instrument 10 into operative engagement with its respective nozzle coupling 44. This can readily be achieved in a manner which advantageously reduces the likelihood of damage to the tool element associated with the instrument, and can also reduce likelihood of a needle-stick injury to the personnel/operative involved in the cleaning process. All engagement by such personnel/operative with the instrument can occur at the distal end of the cylindrical handle body 16 remote from the tool element 28, 30.

The invention proves advantageous in reducing the amount of manipulation required for a cleaning procedure and therefore advantageously reduces the likelihood of damage to the tool and injury to an operative. The invention therefore enhances the possibility for reuse of such instruments leading to both cost, and environmental, savings.

It should of course be appreciated that the invention is not restricted to the details of the foregoing embodiment. For example, the invention can be provided with any form of squeeze handle ophthalmic device arranged to provide any particular form of surgical function/operation. As such the invention is not restricted to the details of the particular tool of the illustrated embodiments, and can be configured, and indeed configure for use with, any form/type of tool as required.

Also, although the above-mentioned example is described very much in terms of cleaning/flushing an instrument/tool, it will be appreciated that the invention also provides for the arrangement and use of the conduit for the delivery of fluid substance into the eye at optimal location for maximal effect and minimal required amount/concentration. Examples of such fluid substances include, vegetative dyes (“Vision Blue” from DORC), also known as methylene blue, antibiotics, steroids, and clotting agents. Such examples are “intracameral”, in so far as they are injected into the anterior chamber of the eye, but the invention is not restricted to examples of that nature.

The invention finds advantageous use irrespective of the nature of the tool provided at the tool portion.

1. A squeeze handle for an ophthalmic instrument and having an elongate body with resiliently-biased squeeze elements, wherein a conduit is provided within the elongate body to allow for fluid communication between an opening in an outer wall of the elongate body and a tool of the instrument when in operative engagement with the handle and the squeeze elements.

2. The squeeze handle as claimed in claim 1, wherein the opening is provided in a region of an end of the elongate body to be distal the tool.

3. The squeeze handle as claimed in claim 1, wherein the conduit extends along at least the substantial length of the elongate body.

4. The squeeze handle of claim 1, wherein the opening has an engagement formation configured to engage with an output of a fluid delivery arrangement.

5. The squeeze handle of claim 1, wherein the conduit is configured to deliver fluid to within at least part of the tool of the instrument.

6. The squeeze handle of claim 1, wherein the conduit is configured to be contiguous with, and deliver fluid to, a conduit within the tool of the instrument.

7. The squeeze handle of claim 6, wherein the conduit in the body is configured to be coaxial with the conduit within the tool of the instrument.

8. The squeeze handle of claim 1, wherein the elongate body is arranged for replaceable mounting of another tool.

9. The squeeze handle of claim 1, wherein the tool is integral with the elongate body.

10. The squeeze handle of claim 1, wherein the handle includes means for receiving a cleaning fluid.

11. The squeeze handle of claim 10, wherein the means for receiving the cleaning fluid includes means for receiving cleaning fluid to flush the instrument.

12. The squeeze handle of claim 1, including means for receiving a fluid substance and means for delivering the fluid substance via the instrument into a patient's eye.

13. The squeeze handle of claim 1, including a tool mount portion in fluid communication with the elongate body for receiving the tool.

14. The squeeze handle of claim 13, wherein at least part of the tool mount portion is movable relative to the elongate body for actuation of the tool.

15. The squeeze handle of claim 13, wherein the tool mount portion is a unitary member arranged to be movable relative to the elongate body for actuation of the tool.

16. The squeeze handle of in claim 13, wherein the tool mount is received in the conduit of the elongate body and itself has a conduit in communication with the conduit of the elongate body to define a passage for fluid to the tool.

17. The squeeze handle of claim 16, including means for securing of the tool to the tool mount or elongate body and within the passage to allow for relative movement between the tool and at least part of the tool mount.

18. The squeeze handle of claim 1, wherein a width of a path for the fluid communication decreases at least once in the direction of fluid flow.

19. The squeeze handle of claim 18, wherein the at least one decrease in the path width is in the region of the tool.

20. A method of cleaning an ophthalmic instrument having resiliently biased squeeze elements on an elongate handle body, the method including:
a. introducing, by way of an opening provided in an outer wall of the elongate body, fluid under pressure into a conduit provided within the elongate body; and,
b. allowing for flow of the fluid along the conduit to a tool in operative engagement with the elongate handle and squeeze elements; for
c. cleaning the tool.

21. The method as claimed in claim 20, including directing the fluid introduced into the conduit into an inner part of the tool.

22. The method as claimed in claim 21, including flushing the tool by way of the fluid.

23. The method as claimed in claim 21, including mounting the ophthalmic instrument by way of the opening in the elongate body onto a fluid delivery arrangement.

24. The method as claimed in claim 21, including mounting the ophthalmic instrument onto a nozzle of a fluid delivery arrangement.

25. The method of claim 21, including forming at least part of an automated cleaning process.

26. A method for the introduction of a fluid substance into a patient's eye, by way of an ophthalmic instrument having resiliently biased squeeze elements on an elongate handle body and including:
a. introducing, by way of an opening provided in an outer wall of the elongate body, the fluid substance into a conduit provided within the elongate body; and,
b. allowing for flow of the fluid along the conduit to a tool in operative engagement with the elongate handle and squeeze elements; for
c. delivery of the fluid substance to the patient's eye via the tool.

27. An ophthalmic instrument fluid introduction apparatus, including:
a squeeze handle having an elongate body with resiliently-biased squeeze elements, wherein a conduit is provided within the elongate body to allow for fluid communication between an opening in an outer wall of the elongate body and a tool of the instrument when in operative engagement with the handle and the squeeze elements; and
a fluid delivery mechanism having at least one nozzle arrange to engage with the opening in the body of the squeeze handle.

28. The apparatus of claim 27, wherein the at least one nozzle is arranged for mounting of the ophthalmic instrument within the apparatus.

29. The apparatus of claim 28, including a plurality of nozzles for receipt of a plurality of ophthalmic instruments.

30. The apparatus of claim 29, including a housing within which the nozzles are located.

31. The apparatus of claim 30, arranged with control functionality allowing for an at least part-automated cleaning process.
